(19) United States
(12) Patent Application Publication
(10) Pub. No.: US 2011/0054338 A1
(43) Pub. Date: Mar. 3, 2011

(54) DEVICE, SYSTEM AND METHOD FOR MONITORING HEAT STRESS OF A LIVESTOCK ANIMAL

(75) Inventors: Raphael LINKER, Yeshuv Eshhar- Doar-Na Misgav (IL); Vladimir Kushnir, Rosh Pina (IL)

(73) Assignee: Technion Research & Development Foundation Ltd., Haifa (IL)

(21) Appl. No.: 12/616,882

(22) Filed: Nov. 12, 2009

Related U.S. Application Data
(60) Provisional application No. 61/114,605, filed on Nov. 14, 2008.

(51) Int. Cl. A61B 5/08

(52) U.S. Cl. 600/529

(57) ABSTRACT

A method of estimating heat stress of livestock animals. The method comprises capturing a sequence of images each depicting a thorax of a livestock animal during a monitoring period, analyzing the sequence of images to detect a movement frequency of the thorax during the monitoring period, computing a breathing rate of the livestock animal as a function of the movement frequency, and estimating a heat stress status for the livestock animal according to the breathing rate.

### RELATED APPLICATION/S

This application claims the benefit of priority from U.S. Provisional Patent Application No. 61/114,605 filed on Nov. 14, 2008. The content of the above application is incorporated by reference as if fully set forth herein.

### FIELD AND BACKGROUND OF THE INVENTION

The present invention, in some embodiments thereof, relates to method and system of monitoring heat stress and, more particularly, but not exclusively, to method and system of monitoring heat stress of livestock animals.

Milk producers can mitigate many negative effects of summer heat with proper livestock animal comfort, nutrition and reproductive management. When a livestock animal is exposed to heat stress, not only does its milk production suffer, but its ability to feed properly, reproduce, and maintain its auto-immune system may all become compromised. Heat stress can be triggered by temperature, humidity, or both.

Biological response to other forms of stress such as crowding, poor ventilation, poor footing and poor stall design have not been well established for dairy cows. First signs of heat stress can already be seen at 20° C., with cows sweating and breathing fast. Milk yield can decrease by about 10 percent. Studies have shown that heat stress during late gestation reduces calf birth weight and subsequent milk production, see Dunham, D. et al, Coping with summer weather, Ominski, K. H., Kennedy, A. D., Wittenberg, K. M., Moshtaghi Nia, S. A. (2002), Physiological and production responses to feeding schedule in lactating dairy cows exposed to short-term, moderate heat stress. Journal of Dairy Science 85: 730-737, Legates, J. E., Farthing, B. R., Cascade, R. B., Barada, M. S. (1991), Body temperature and respiratory rate of lactating dairy cattle under field and chamber conditions. Journal of Dairy Science 74: 2491-2500, Meyer, M. J., Smith, J. F., Hamer, J. P., Shirley, J. E., Titgemeyer, E. C., Brouk, M. J. (2002) Performance of lactating dairy cattle in three different cooling systems, Applied Engineering in Agriculture 18: 341-345, and Hillman, P. E., Lee, C. N., Willard, S. T. (2005). Thermoregulatory responses associated with lying and standing in heat-stressed dairy cows. Transactions of the ASAE 48: 795-80 1, which are incorporated herein by reference.

In order to avoid heat stress, livestock animals are kept in facilities which are cooled by cooling systems that includes fans, droppers, sprinklers, and/or the like. Most of the cooling systems are operated by temperature and/or humidity sensors.

During the last years, systems and methods for estimating the affect of heat on the physiological state of livestock animals have been developed.

For example U.S. Pat. No. 6,059,733, filed on 23 Jul. 1998 describes a method of determining a physiological state of a ruminant animal monitors the core body temperature of the ruminant animal. A bolus including a temperature sensor and a transmitter is placed within a stomach of the ruminant animal. A plurality of temperatures are sensed within the stomach using the sensor, with each temperature representing a temperature at a respective discrete point in time over a time period. A plurality of air-borne signals are transmitted to a remote receiver using the transmitter, with each air-borne signal representing at least one of the sensed temperatures. The plurality of temperatures are mathematically analyzed at discrete points in time over the time period using the remote receiver. The physiological state of the ruminant animal is determined using the mathematically analyzed temperatures.

Another example is provided in U.S. Pat. No. 7,234,421, filed on Mar. 20, 2002 that describes an animal data gathering device comprise: a radio transmitter and receiver, a processor for controlling the operation of the device, and memory for storing, information including a first unique identifier associated with the device. The processor is arranged to transmit a signal, by means of the radio transmitter, and to receive, by means of the radio receiver, one or more signals, each representing a second unique identifier from other devices and the processor is arranged to store in the memory each second unique identifier. Thus a record is kept of all radio devices with which the device has come into radio contact.

### SUMMARY OF THE INVENTION

According to some embodiments of the present invention there is provided a method of estimating a heat stress of a livestock animal. The method comprises capturing a sequence of images each depicting a thorax of a livestock animal during a monitoring period, analyzing the sequence of images to detect a movement frequency of the thorax during the monitoring period, computing a breathing rate of the livestock animal as a function of the movement frequency, and estimating a heat stress status for the livestock animal according to the breathing rate.

Optionally, the livestock animal is a cow.

Optionally, the analyzing comprises identifying a contour of the livestock animal in at least some of the sequence of images, identifying, in the at least some images, a plurality of instantaneous widths of the thorax using the contour, and calculating the movement frequency according to the plurality of instantaneous widths.

More optionally, the analyzing further comprises at least one of rotating and translating each one of the at least some images to a common orientation.

More optionally, the computing is performed by applying a fast Fourier transform (FFT) on a vector of the plurality of instantaneous widths.

More optionally, the contour is identified by a snake algorithm. Optionally, the method is iteratively repeated during a period of at least 12 hours.

Optionally, the method further comprises preprocessing the sequence of images to remove artifacts therefrom.

Optionally, the method further comprises preprocessing the sequence of images to remove artifacts therefrom.

Optionally, the method further comprises detecting a placing of the livestock animal in a stall by processing the sequence of images and before the analyzing.

Optionally, the livestock animal is free to more in a stall during the capturing.

Optionally, at least some of the sequence of images depicts the livestock animal in a plurality of places of a stall during the capturing.

According to some embodiments of the present invention there is provided a device of monitoring a heat stress of a livestock animal. The device comprises an image sensor which captures a sequence of images of a thorax of a livestock animal during a monitoring period, a processing unit which analyzes the sequence of images to detect a movement frequency of the thorax, computes a breathing rate of the livestock animal accordingly, and estimates heat stress status of the livestock animal according to the breathing rate, and an output unit which outputs a message indicative of the heat stress status.

Optionally, the message is an alarm.

Optionally, the device further comprises a memory for storing at least one reference value indicative of an exemplary breathing rate of a livestock animal with a heat stress, the processing unit estimates the heat stress status by comparing between the reference value and the breathing rate.

Optionally, the device further comprises an additional sensor for measuring at least one surrounding condition in proximity to the livestock animal, the processing unit estimates the heat stress status according to a function of the at least one surrounding condition and the breathing rate.

According to some embodiments of the present invention there is provided a system of monitoring a heat stress of a plurality of livestock animals. The system comprises a plurality of image sensors separately each placed in a respective of a plurality of stalls and captures a sequence of images of a thorax of a respective of the plurality of livestock animals, placed in the respective stall, during a monitoring period, a processing unit which analyzes each the sequence of images and computes accordingly, for each the livestock animal, a breathing rate, the processing unit estimates a heat stress status of each the livestock animal according to the breathing rate, and an output unit which outputs a message indicative of the estimation.

Optionally, the system further comprises a display for presenting the message.

Optionally, the system further comprises a communication interface for forwarding the message to a member of a group consisting of: a remote server, a storage unit, a cellular device, and a message server.

Optionally, the system further comprises a communication interface for controlling, according to the message, a member of a group consisting of: a cooling system, a livestock milking equipment, a livestock feeding equipment, a livestock watering equipment, a livestock manure removal equipment, livestock bedding, and/or livestock exercise equipment.

Optionally, the message is used for producing a report indicative of the heat stress status of each the livestock animal in any selected period during their stay in the plurality of stalls.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volitile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a system of monitoring a livestock animal, such as a cow in an individual stall of a stall barn, according to some embodiments of the present invention;

FIG. 2 is a flowchart of a method of a detecting a heat stress of a livestock animal, according to some embodiments of the present invention;

FIG. 3 is a flowchart of a process for estimating a breathing rate of a livestock animal as a function of the movements of the thorax, according to some embodiments of the present invention;

FIGS. 4A-4C respectively depict an exemplary frame of a monitored livestock animal, a binarized version of exemplary frame, and a rotated and translated version of the exemplary frame, according to some embodiments of the present invention;

FIGS. 5A and 5B respectively depict an exemplary initial contour which is placed at the center of a frame depicting an livestock animal and an exemplary final contour which has been iteratively fitted on the livestock animal, according to some embodiments of the present invention;

FIG. 6 is a graph that depicts the instantaneous width of the thorax of a pictured livestock animal during a plurality of instants of a monitoring period, according to some embodiments of the present invention;

FIG. 7 is a graph that depicts a derivative of the data presented in FIG. 6 which is generated by an a Fourier transform (FFT), according to some embodiments of the present invention; and

FIG. 8 is a schematic illustration of a system for monitoring a plurality of livestock animals hosted, optionally separately, in a plurality of individual stalls, according to some embodiments of the present invention.

### DESCRIPTION OF EMBODIMENTS OF THE INVENTION

The present invention, in some embodiments thereof, relates to method and system of monitoring heat stress and, more particularly, but not exclusively, to method and system of monitoring heat stress of livestock animals.

According to some embodiments of the there is provided a method, a device, and a system of a estimating a heat stress of a livestock animal, such as a cow that is placed in a stall. The estimation is based on an analysis of a sequence of images that depicts a movement frequency of a thorax of a livestock animal during a monitoring period. The images are optionally taken from above, depicting the back of the livestock animal. The sequence of images may be captured using a regular video camera. The sequence is than analyzed to detect the movement frequency of the thorax of the monitored livestock animal during the monitoring period. This analysis allows computing the breathing rate of the livestock animal as a function of the movement frequency. The breathing rate is now used for estimating the heat stress status for the livestock animal.

Optionally, the movement frequency is detected extracting the width of the thorax of the livestock animal from some or all of the images. The extracted instantaneous widths are concatenated to generate a vector that represents a change of the width during the period in which the sequence of images is captured. Now the breathing rate may be calculated as a function of this vector, for example by applying a fast Fourier transform (FFT).

Optionally, the estimation of the heat stress status is used for controlling a cooling system for reducing the heat stress of the livestock animal. Other systems may be controlled according to the heat stress status, for example as described below.

According to some embodiments of the present invention, a system of monitoring a heat stress of a plurality of livestock animals. The system comprises a plurality of image sensors which are placed in a plurality of stalls and captures image sequences of the livestock animals. The system further comprises a processing unit that analyzes the outputs of the image sensors. The processing unit estimates the heat stress status of each one of the livestock animals which are placed in the plurality of stalls by this analysis. The system further includes an output unit that outputs a message or a report indicative of the estimation. In such a manner, the heat stress status of a plurality of livestock animals may be monitored simultaneously. Optionally, energy detected for cooling the livestock animals is allocated according to the outputted message or report. For example, while cooling systems are activated in stalls in which animals with an estimated stress heat have been detected, other cooling systems are deactivated.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

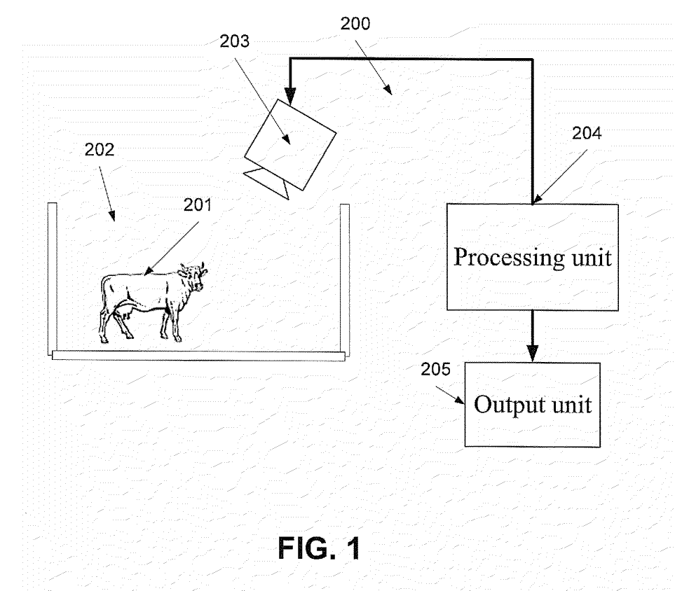

Reference is made to FIG. 1, which is a schematic illustration of a device 200 of monitoring a livestock animal 201, such as a cow, placed in an individual stall 202 of a stall barn, according to some embodiments of the present invention. For brevity, a livestock animal means a non-human mammal optionally capable of producing milk, such as cows, sheep, goats, horses, donkeys, bison, buffalo, antelope, elk, deer, llamas, pigs, and camels, among others. A cow means a female of the family Bovidae, including for example, buffalo, bison, wild and domesticated cattle, sheep, goats, and water buffalo.

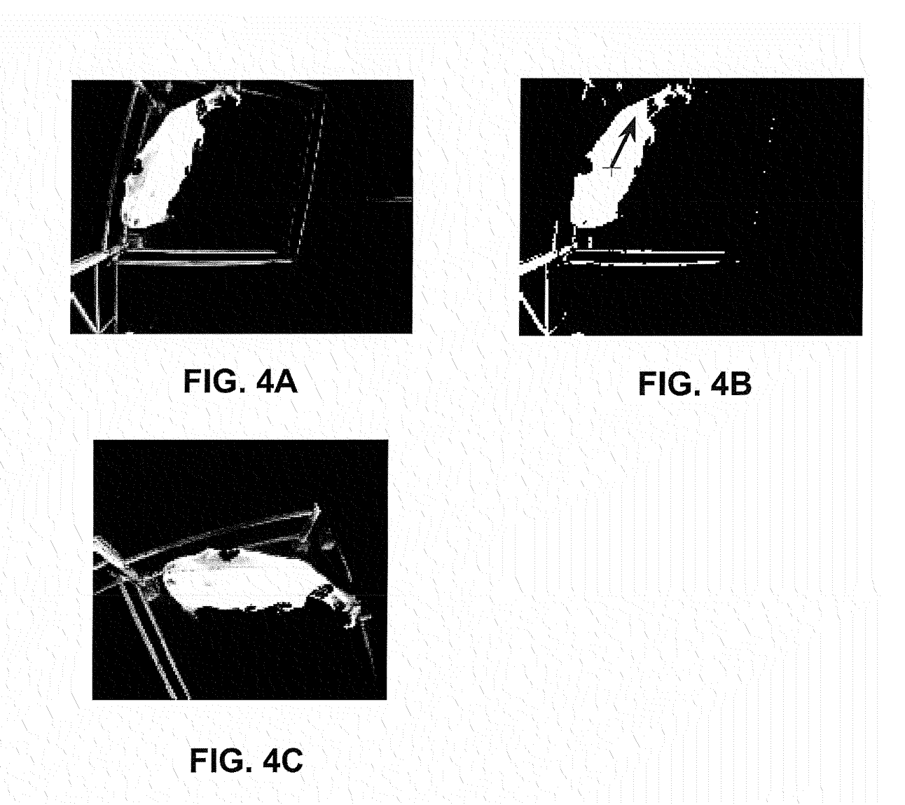

The device 200 includes an image sensor 203, such as a charge coupled device (CCD) based sensor or a complementary metal-oxide-semiconductor (CMOS) based sensor, for example a standard 492*658 pixels industrial RGB video camera (Blaster Scout GigE) equipped with a. TF2.8 DA-8 lens (Navitar). The image sensor 203 is connected, wirelessly or via a cable, such as an Ethernet cable, to a processing unit 204, such as a microprocessor, a computer, and a digital signal processing (DSP). The processing unit 204 detects the presence, the absence, and/or the severity of heat stress of the livestock animal 201 by analyzing the sequence of images, for example as described below. The processing unit 204 is optionally located outside of the stall barn. The processing unit 204 is connected to an output unit 205 that outputting the presence, the absence, and/or the severity of heat stress. For brevity, such outputting includes forwarding the data to a remote server, presenting the data to a user, forwarding the data to a storage device, a cellular device, and a message server and/or activating an alarm. The image sensor 203 is place to capture a sequence of images of the livestock animal 201, optionally at a rate of 30 frames per second. Optionally, the image sensor 203 is placed to capture the upper side of the thorax of the livestock animal 201, for example as depicted in FIGS. 4A-4C. Optionally, the image sensor is attached to the roof of the individual stall 202, to a stand, and/or to the upper segments of the individual stall's walls, for example about 4 meters above the floor of the individual stall 202.

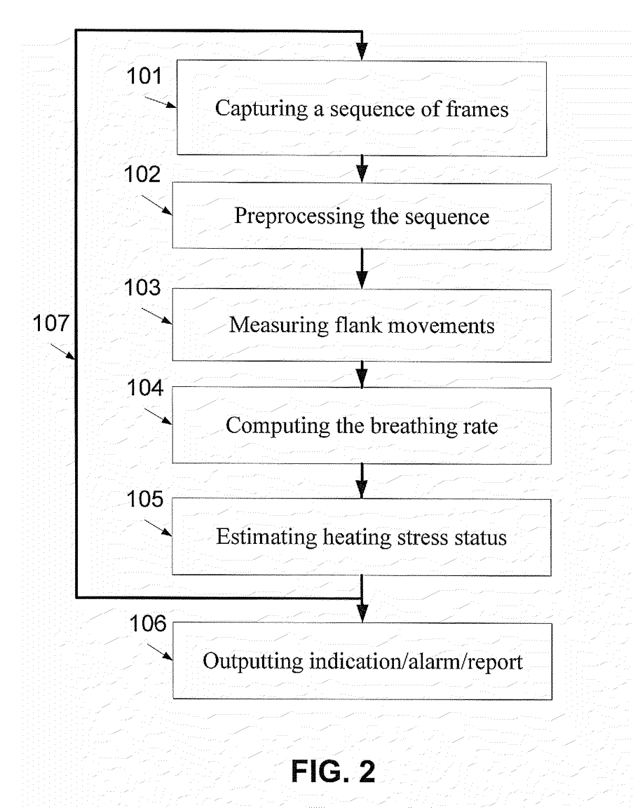

Reference is now also made to FIG. 2, which is a flowchart of a method of a detecting a heat stress of a livestock animal, according to some embodiments of the present invention.

In use, as shown at 101, the image sensor 203 captures images, which may be referred to herein as frames, of the space of the individual stall 202.

Optionally, as shown at 102, the captured images are preprocessed for reducing noises and/or emphasizing potential lines and curves which may be part of the contour of the livestock animal 201. The noise reduction may be performed by using filters such as salt and pepper noise filters, Minimum filters, Maximum filters and/or Median filters, for example by an erosion-dilation process. Optionally, the potential lines and curves are emphasized by convoluting designated masks which correlate with the lines and curves.

Now, as shown at 103, the captured sequence of images is analyzed to measure the frequency of the movements of the thorax of the pictured livestock animal 201 during a monitoring period. As shown at 104, such measurements allow computing the breathing rate of the livestock animal.

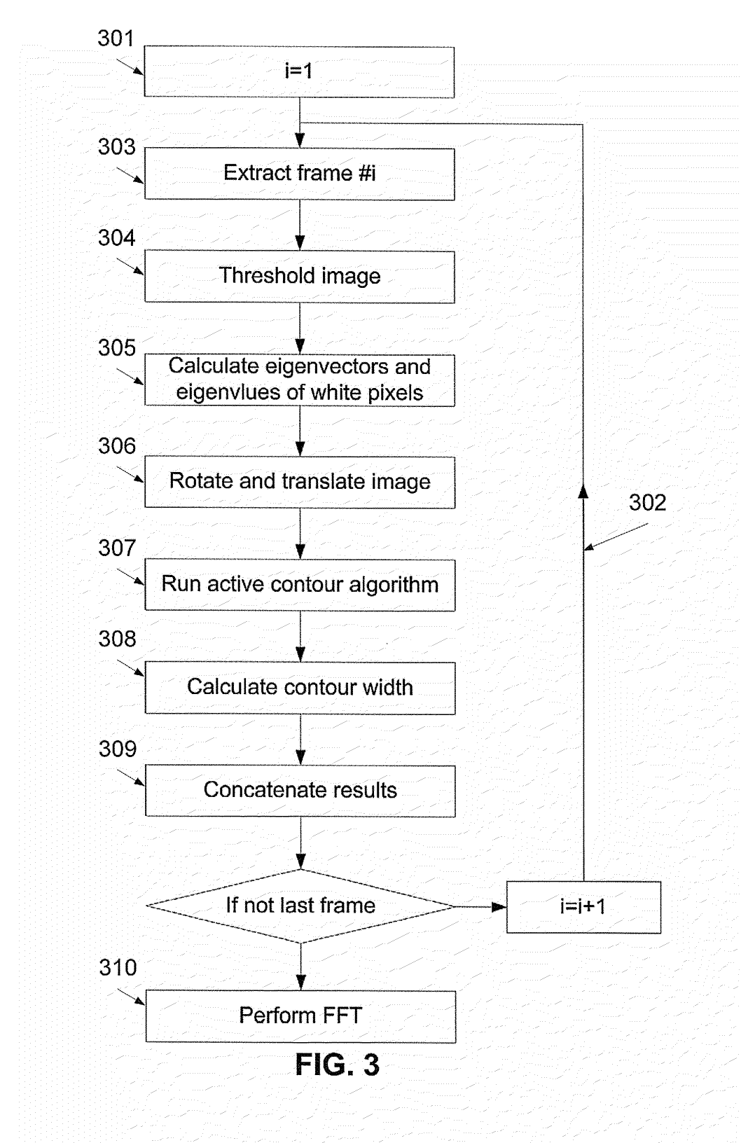

Reference is now made to FIG. 3, which is a flowchart of a process for estimating a breathing rate of a livestock animal 201 as a function of the movements of the thorax, as shown by blocks 103 and 104 of FIG. 2, according to some embodiments of the present invention. The process is based on a sequence of i frames, which are captured as described above and depicts the livestock animal 201 within the individual stall 202. The process includes an iterative sub process 302 in which each frame is separately analyzed to extract the instantaneous width of the thorax of the livestock animal 201 at the moment frame i has been captured. The instantaneous width is optionally extracted by identifying the contour of the livestock animal 201 and measuring the distance between the left and right sides of the contour, at a lateral bisector that is estimated to divide the livestock animal's flanks.

First, an index i is reset to 1, as shown at 301. Then, the iterative sub process 302 is initiated. First, as shown at 303, frame i is selected. An example for such a frame is provided in FIG. 4A that depicts a cow in an individual stall of a cowshed.

As the livestock animal 201 may move freely within the individual stall 202, the figure thereof has to be roughly identified so as to allow rotating and/or translating the frame such it is brought to a standard position, for example by translation and/or rotation operations. In this manner, a common initial contour may be used for all the images as they are all centered according to the same rules. For example, if the livestock animal 201 is a cow, the common initial contour may be an elliptic initial contour of predetermined size. In order to calculate the required translation-rotation move, as shown at 304, frame i is converted to a binary form, for example by a thresholding operation that is based of the color of the livestock animal 201. For example, if the livestock animal 201 is a cow, such as a Holstein-Friesian breed cow, the thresholding is performed on the basis of white color. If the cow is a Brown Swiss breed or Ayrshire breed, the thresholding may be performed on the basis of brown color. Such adaptations may be performed for other breeds, for example Guernsey, Tharparkar, Jersey, and Milking Shorthorn. The thresholding allows creating a binary frame in which white and/or black pixels correspond to the livestock animal 201 while others may correspond to the individual stall 202. For example, FIG. 4B is a binarized version of the frame that is depicted in FIG. 4A.

Optionally, the device 200 is calibrated before the monitoring is initialized. The calibration may set values for the thresholding. The values may be set manually, for example inputted by an operator and/or automatically, for example by capturing an image of the livestock animal and determining its color and/or hue.

The binary image allows creating a matrix that contains coordinates of livestock animal pixels. The binary image is optionally segmented according to the livestock animal pixels so as to differentiate a livestock animal segment from its surrounding. As shown at 305, the eigenvectors and eigenvalues of the livestock animal pixels are calculated. As shown at 306, the rotation/transformation of the segment of the livestock animal pixels, referred to herein as a livestock animal segment, is calculated according to the eigenvector associated with the largest eigenvalue of a co-variance matrix that is based on the aforementioned matrix. Now, frame i is translated to the a-priori defined location by mean removal and bias addition according to the orientation of the rotated livestock animal segment. For example, FIG. 4C is a rotated and translated version of FIG. 4A.

Now, as shown at 307, the contour of the livestock animal 201 is calculated. Optionally, the contour is calculated according to an active contour process that iteratively converges from an initial contour toward the actual contour of the object. It should be noted that as the image is rotated and translated before the active contour process begins, at least part of the figure of the livestock animal in the frame i is included in the initial contour. Optionally, the active contour is based on a snake algorithm, for example as described in Davis, P. F., and J. A. Marchant. 1993. Pig image outlining using artificial neuron parameters in the snake contour method. Computers and Electronics in Agriculture 8: 277-292; Marchant, J. A., and C. P. Schofield. 1993. Extending the snake image processing algorithm for outlining pigs in scenes. Computers and Electronics in Agriculture 8: 261-275; Marchant, J. A., and C. M Onyango. 1995. Fitting grey level point distribution models to animals in scenes. Image and Vision Computing 3: 3-12; and Onyango, C. M, J. A. Marchant, and B. P. Ruff 1995, Model based location of pigs in scenes, Computers and Electronics in Agriculture 12: 261-273, which are incorporated herein by reference. In this process, which is based on differential geometry, an initial contour, which is set according to the aforementioned rotation and translation, and a cost function are set. It should be noted that choosing an initial contour relatively close to the actual contour of the object ensures a faster convergence. Now, the initial contour is iteratively deformed, for example expended and/or contracted, until the cost function is minimized. An example for an implementation of such a process is available in the Matlab toolbox which has been developed by E. Debreuve, see Debreuve, E., M. Barlaud, J. P. Marmorat, and G. Aubert. 2006. Active contour segmentation with a parametric shape prior: Link with the shape gradient. Proceedings of the International Conference on Image Processing (ICIP), October 8-11, Atlanta, Ga. (GA), USA and http://www.mathworks.com/matlabcentral/fileexchange/authors/24536, which are incorporated herein by reference.

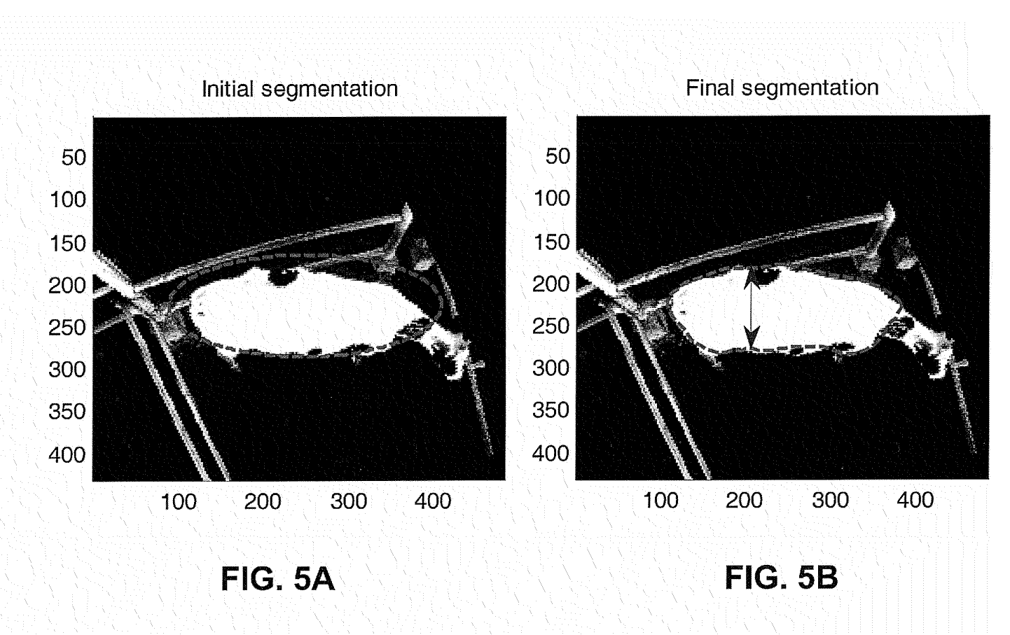

For example, exemplary initial contour and exemplary final contour are respectively shown at FIGS. 5A and 5B. As depicted in these figures, the region of interest of the final contour fits closely to the actual contour of the livestock animal. Optionally, as shown in FIGS. 5A and 5B, the initial contour is defined so as to exclude the head of the livestock animal from being identified as part of the livestock animal segment.

Now, after the contour has been obtained, the instantaneous width of the contoured object, namely the livestock animal, at a certain section may be determined, as shown at 308. This instantaneous width is indicative of the current volume of the livestock animal's thorax, a volume that is changed with respect to the stage of the breathing cycle of the livestock animal. When the livestock animal inhales, the volume increases and when the livestock animal exhales, the volume decreases. Optionally, the instantaneous width is determined by measuring the length of a line that divides the livestock animal segment in a ratio of about 1:2 along its longitudinal axis where the segment that has a narrower end is the large segment. For example, see the instantaneous width that is indicated by a double headed arrow in FIG. 5B. This double headed arrow depicts an actual contour of the cow, a contour that has been calculated based on the initial contour which is depicted in FIG. 5A.

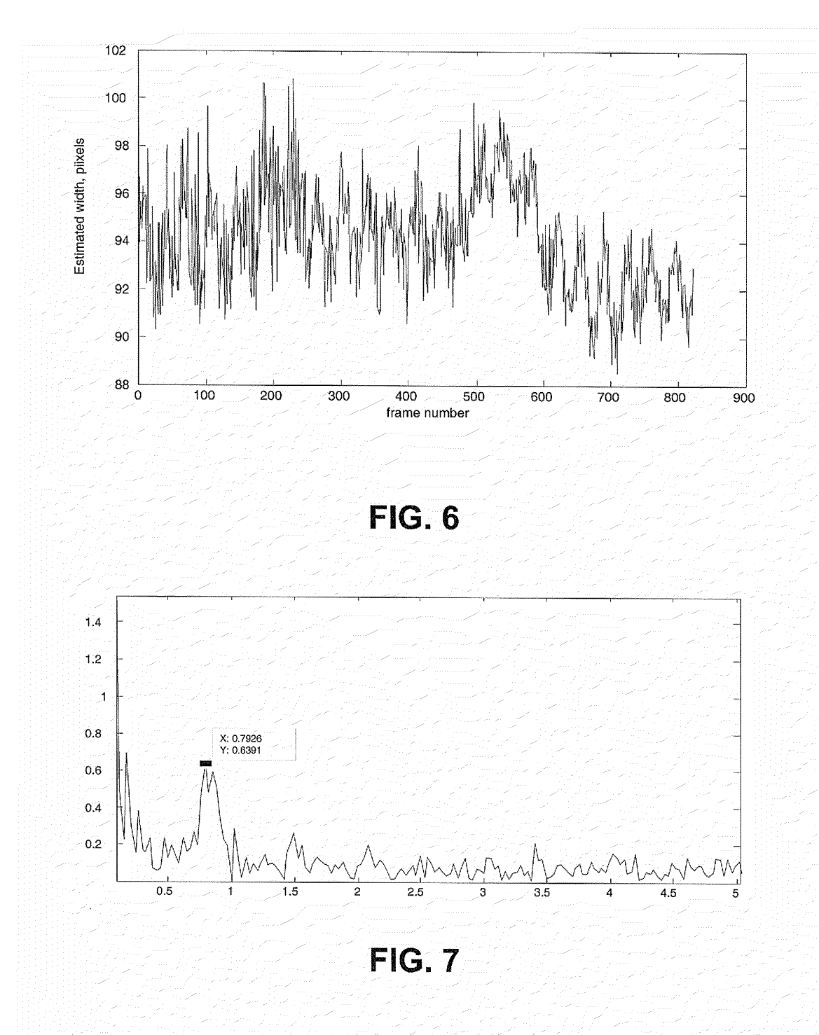

Now, as shown at 309, the measured instantaneous width is added to a width vector that concatenates the instantaneous widths which are iteratively measured as shown at 302. FIG. 6 is a graph that depicts the instantaneous width of the thorax of the pictured livestock animal during a monitoring period. Axes X and Y of FIG. 6 indicate frame numbers and widths depicted in these frames.

As depicted in FIG. 6, the correspondence of the graph to a breathing cycle is clear. The graph has a shape of a low-frequency sinusoidal signal that corresponds with the sinusoidal nature of a breathing cycle.

As the width vector is created based on data that is gathered during a period of one or more breathing cycles, noise and/or sudden jumps, which are caused to movement of the animal and/or uncontrolled muscle contractions, does not substantially effect the trend which may be reflected from the analysis of the width vector, for example as described below.

Now, as shown at 310, an FFT is applied on the width vector, yielding a derivative vector that reflects the breathing rate. For example, FIG. 7 is a graph that depicts an FFT of the width vector depicted in FIG. 6. As depicted in FIG. 7, the breathing rate is identified as approximately 0.8 Hz or 48 breaths per minute.

Reference is now made, one again, to FIGS. 1 and 2. After the breathing rate has been computed, for example according to the process that is depicted in FIG. 3, the heat stress status of the monitored livestock animal is estimated accordingly, as shown at 105. As used herein, a heat stress status means a presence, an absence, and/or a level of severity of a heat stress at the monitored livestock animal. Optionally, the device 200 includes a memory unit for storing breathing rate values and/or patterns, each associated with a definition that is indicative of the heat stress status.

The computed breathing rate is compared with the stored breathing rate reference values and/or patterns. This comparison allows diagnosing the heat stress status, for example whether the livestock animal is in a heat stress or not and/or what is the severity of the heat stress.

Optionally, as shown at 107, this estimation is performed iteratively, continuously and/or intermittently. For example, the device 200 may perform the estimation cycle that is depicted in blocks 101-105 every few minutes or hours. The estimation cycles may be repeated during monitoring periods, of hours and/or days and/or continually. For example, the estimation cycles may be repeated during periods of more that 12 hours or 24 hours. Optionally, the monitoring period is initiated whenever the livestock animal is placed in a respective stall. Optionally, the detection of such presence is performed by analyzing the sequence of images which are captured by the image sensor 203. Such an analysis may be performed by known object and/or movement detection image processing methods which are known in the art. Optionally, the device may perform the estimation cycle in response to the detection of a potential heat stress that is valuated in response to a measurement of one or more additional sensors which measure surrounding conditions in proximity to the livestock animal, for example temperature sensors, humidity sensors and the like. In such an embodiment, an alarm may be outputted only when the estimation indicate on a heat stress.

As shown at 106, the estimation may be outputted and/or trigger the outputting of a heat stress alarm, a heat stress notification and/or a heat stress status report. For example, the estimation may be forwarded to a central unit and/or recorded for future analysis. Optionally, an alarm is activated if a heat stress is diagnosed and/or if the severity thereof is above a certain level. Optionally, the alarm is a message that is sent to one or more user, for example as a short message service (SMS) and/or an instant message (IM).

Reference is now made to an experiment made according to the aforementioned method. The experiment was performed using a standard 492*658 pixels industrial RGB video camera (Blaster Scout GigE) equipped with a TF2.8 DA-8 lens (Navitar) that was placed 4 meters above a barn's floor. The imaged animals were cows which are mostly white, such as the one shown in FIG. 4A. To ensure that most of the video sequences would contain an animal and not just bare soil, a 3 meters by 3 meters temporary enclosure was placed underneath the camera and an animal were placed in the enclosure before recording each sequence. The cow was not restrained and could move and turn freely inside the fenced area.

Table 1 below summarizes exemplary results obtained by capturing five sequences of images (recorded at a frame rate of 30 frames per second) depicting a livestock animal and analyzing them according to the method described above in relation to FIG. 2. The table exemplifies that in all cases the breathing rates estimated by implementing the method are similar to the breathing rates which have been respectively measured visually or expected based on the weather conditions.

TABLE 1

| | Breathing rate | Breathing rate |
|---|---|---|
| | (breath/minute) | (breath/minute) |
| Sequence | estimated by the | determined visually or expected |
| number | described method | based on weather conditions |
| 1 | 48 | 52 |
| 2 | 44 | 52 |
| 3 | 28 | 32 |
| 4 | 21 | 20 * |
| 5 | 18 | 20 * |

where * denotes a breathing rate estimated based on weather conditions.

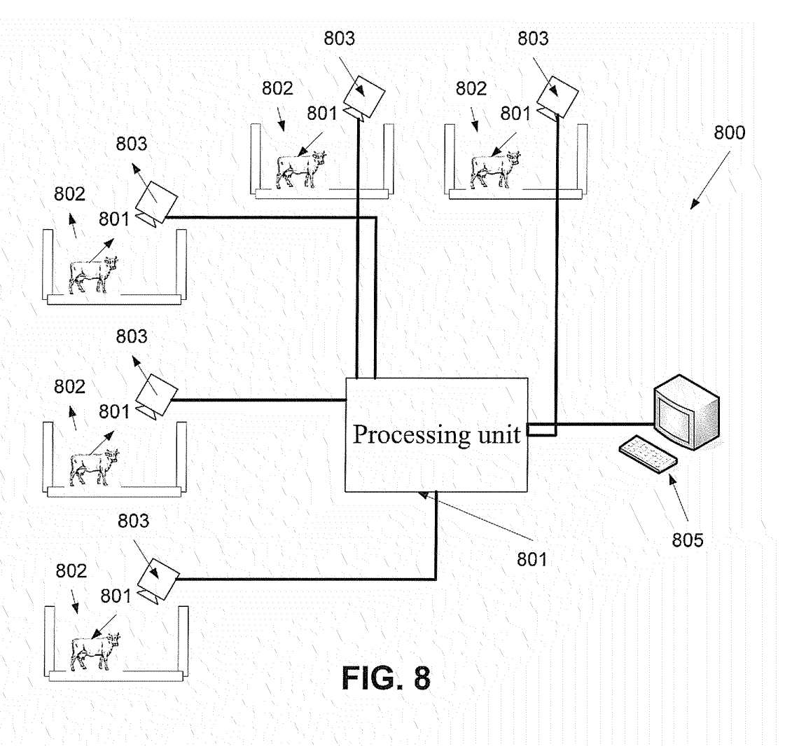

Reference is now made to FIG. 8, which is a schematic illustration of a system 800 for monitoring a plurality of livestock animals 801 which are hosted, optionally separately, in a plurality of individual stalls 802, according to some embodiments of the present invention.

The system includes a plurality of image sensors 803, each installed in a different individual stall 802. The individual stalls 802 may be all in a common stall barn or structure and/or spread in a plurality of barns or structures. Optionally, each image sensor 803 is as image sensor 203 that is depicted in FIG. 1. The system 800 further includes a processing unit 804 that is optionally similar to processing unit 204 depicted in FIG. 1. However, the processing unit 804 is defined for estimating the heat stress status for each one of the livestock animals 801 in the different individual stalls 802. The processing unit 804 receives the sequence of images from each one of the image sensors 803 and analyses it, for example as described above in relation to FIGS. 1-3. In such a manner, the processing unit 804 monitors the stress heat of each member of a certain livestock simultaneously, optionally continually during long motoring periods of few hours, few days, few weeks and the like. This ability allows the system 800 to control a common resource that is used for the livestock, for example the operation of a cooling system. The system 800 can control the cooling system to cool, or cool further, stalls that accommodate animals 801 with heat stress and/or animals 801 with a heat stress level that is indicative of developing a heat stress. The system 800 may manage an energy budget that is allocated for cooling according to the heat stress status estimation, directing energy for cooling stalls that accommodate animals 801 with heat stress and/or animals 801 with a heat stress level that is indicative of developing a heat stress.

Optionally, the system 800 includes a man machine interface (MMI) 805, such as a touch screen and a display and a keyboard, which allows a user to monitor separately each member of a livestock. For example, the MMI is designed to present a graphical user interface (GUI) that presents a separate indication for each member of the livestock. Optionally, an alarm that is indicative of a high level of heat stress, as described above, may be presented using this MMI. For clarity, a livestock means fully or partially livestock animals 801 and milk-producing animals, for example. Livestock does not include microbes, fish, or humans, for example. Optionally, the system 800 allows controlling animal husbandry, such as feeding livestock, raising livestock, providing care for livestock, and obtaining livestock products, for example. Such an MMI may be connected to the aforementioned the system 800 the device 200, providing similar functions only with respect to a single livestock animal 801.

Optionally, the processing unit 804 automatically or semi automatically, for example by requesting a user input, controls livestock equipment, such as livestock milking equipment, livestock feeding equipment, livestock watering equipment, livestock manure removal equipment, livestock bedding, livestock cooling system, and/or livestock exercise equipment according to the computed stress heat.

It is expected that during the life of a patent maturing from this application many relevant systems and methods will be developed and the scope of the term image sensor and computing unit is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of estimating a heat stress of a livestock animal, comprising:
capturing a sequence of images each depicting a thorax of a livestock animal during a monitoring period;
analyzing said sequence of images to detect a movement frequency of said thorax during said monitoring period;
computing a breathing rate of said livestock animal as a function of said movement frequency; and
estimating a heat stress status for said livestock animal according to said breathing rate.

2. The method of claim 1, wherein said livestock animal is a cow.

3. The method of claim 1, wherein said analyzing comprises:
identifying a contour of said livestock animal in at least some of said sequence of images,
identifying, in said at least some images, a plurality of instantaneous widths of said thorax using said contour, and
calculating said movement frequency according to said plurality of instantaneous widths.

4. The method of claim 3, wherein said analyzing further comprises at least one of rotating and translating each one of said at least some images to a common orientation.

5. The method of claim 3, wherein said computing is performed by applying a fast Fourier transform (FFT) on a vector of said plurality of instantaneous widths.

6. The method of claim 3, wherein said contour is identified by a snake algorithm.

7. The method of claim 1, wherein said method is iteratively repeated during a period of at least 12 hours.

8. The method of claim 1, further comprising preprocessing said sequence of images to remove artifacts therefrom.

9. The method of claim 1, further comprising preprocessing said sequence of images to remove artifacts therefrom.

10. The method of claim 1, further comprising detecting a placing of said livestock animal in a stall by processing said sequence of images and before said analyzing.

11. The method of claim 1, wherein said livestock animal is free to more in a stall during said capturing.

12. The method of claim 1, wherein at least some of said sequence of images depicts said livestock animal in a plurality of places of a stall during said capturing.

13. A device of monitoring a heat stress of a livestock animal, comprising:
an image sensor which captures a sequence of images of a thorax of a livestock animal during a monitoring period;
a processing unit which analyzes said sequence of images to detect a movement frequency of said thorax, computes a breathing rate of said livestock animal accordingly, and estimates heat stress status of said livestock animal according to said breathing rate; and
an output unit which outputs a message indicative of said heat stress status.

14. The device of claim 13, wherein said message is an alarm.

15. The device of claim 13, further comprising a memory for storing at least one reference value indicative of an exemplary breathing rate of a livestock animal with a heat stress, said processing unit estimates said heat stress status by comparing between said reference value and said breathing rate.

16. The device of claim 13, further comprising an additional sensor for measuring at least one surrounding condition in proximity to said livestock animal, said processing unit estimates said heat stress status according to a function of said at least one surrounding condition and said breathing rate.

17. A system of monitoring a heat stress of a plurality of livestock animals, comprising:
a plurality of image sensors separately each placed in a respective of a plurality of stalls and captures a sequence of images of a thorax of a respective of the plurality of livestock animals, placed in said respective stall, during a monitoring period;
a processing unit which analyzes each said sequence of images and computes accordingly, for each said livestock animal, a breathing rate, said processing unit estimates a heat stress status of each said livestock animal according to said breathing rate; and
an output unit which outputs a message indicative of said estimation.

18. The system of claim 17, further comprising a display for presenting said message.

19. The system of claim 17, further comprising a communication interface for forwarding said message to a member of a group consisting of: a remote server, a storage unit, a cellular device, and a message server.

20. The system of claim 17, further comprising a communication interface for controlling, according to said message, a member of a group consisting of: a cooling system, a livestock milking equipment, a livestock feeding equipment, a livestock watering equipment, a livestock manure removal equipment, livestock bedding, and/or livestock exercise equipment.

21. The system of claim 17, wherein said message is used for producing a report indicative of said heat stress status of each said livestock animal in any selected period during their stay in said plurality of stalls.
